(19) United States
(12) Patent Application Publication
(10) Pub. No.: US 2010/0210973 A1
(43) Pub. Date: Aug. 19, 2010

(54) Medical pressure measuring device

(76) Inventors: Joanna Lynn Cecil, Palm Coast, FL (US); Judith Kaye Korbel, New Cumberland, WV (US)

Correspondence Address: THE INVENTORS NETWORK, INC., 332 ACADEMY STREET, CARNEGIE, PA, 15106, US

(21) Appl. No.: 12/378,316

(22) Filed: Feb. 17, 2009

(51) Int. Cl. A61B 5/103

(52) U.S. Cl. 600/592

(57) ABSTRACT

A medical pressure measuring device placed upon a patient's foot for accurately measuring pounds pressure as the patient stands and ambulates includes a flexible wrap having hook and loop fastening connections, a flat load cell sensor on the wrap bottom interconnected to a readily and easily viewable digital display for showing a digital readout of pounds pressure and power for the digital display being provided by a flat lithium nickel size battery and the wrap adaptable for fitting onto adult-size or child-size feet.

### FIELD OF THE INVENTION

The present invention pertains to medical pressure measuring devices, and more particularly pertains to a medical pressure measuring device that accurately measures pounds/kilograms of pressure applied by a patient as the patient attempts to stand and/or ambulate after surgery and as part of the rehabilitation and convalescence.

### BACKGROUND OF THE INVENTION

The post-surgical rehabilitation and convalescence of individuals with lower extremity fractures or injuries requires a number of steps, processes, and devices to accurately assess and measure the progress of the rehabilitation and convalescence. Such lower extremity fractures and injuries can be quite extensive but the primary types of fractures and injuries include hip replacements and hip fractures. In order to promote healing the patient must slowly and very gradually ambulate but not with his/her full weight being applied to the lower extremities. Instead surgeons will give a specific percentage of weight bearing following surgery that is to be applied to the affected lower extremity. The weight bearing that is provided can be in percentages or in the exact amount of pounds the surgeon or physician wants his or her patient to exert upon ambulation. Such percentages will be graduated as full weight bearing upon the injured extremity must be carefully worked up to and cannot be immediately attained as full weight bearing immediately after surgery would most likely result in the damage or re-injury of the extremity. Thus in order to promote correct injury healing the surgeon would order that the weight bearing applied through and to the extremity commence and proceed from 25% to 33% to 50% to 75% of the patient's total body weight before the patient attempts to exert his/her full weight on the extremity.

However, each patient is unique and brings his/her own health history and problems into play during such rehabilitation. Thus, patients with diabetes, for example, often have numbness and tingling in their feet, and this causes them difficulty—and actually impedes—their ability to feel the amount of pressure—the weight bearing—that they are exerting through their feet. Recuperation and healing are hindered if the patient cannot feel his/her feet or the pressure being exerted therethrough. On the other hand, patients with dementia will need to have their recuperation and gait training carefully monitored by the caregiver or physical therapy team. Patients with dementia often have little or no safety awareness or conscious thought of their weight bearing status during recuperative gait training, and thus the caregiver/physical therapy team must carefully monitor the gait training so that the exact amount of pressure is exerted by the patient on the involved extremity. Therefore, it would be desirable for both the patient and the caregiver/therapists/therapy team to have a means, method or device that provides a visual cue or visual feedback regarding the correct and exact amount of pressure being exerted on the involved extremity.

For example, the Goto patent (U.S. Pat. No. 5,362,929) discloses a weight sensor device that includes two piezoelectric devices interconnected to a voltage monitor circuit that determines the weight of an object.

The Burke et al. patent (U.S. Pat. No. 6,223,606 B1) discloses a center of weight sensor that includes a sensor system for use in a vehicle airbag deployment system.

The Yoshimi et al. patent (U.S. Pat. No. 6,280,392 B1) discloses an infant conditioning monitoring system that includes a sensor sheet having a plurality of pressure sensitive load cells for sensing and calculating an infant's breathing signal, sleeping posture and weight data.

The Bechmann patent (U.S. Pat. No. 6,752,028 B2) discloses a load monitoring test device for a patient's foot that includes a ring-shaped curved washer that is snapped into position on the patient's heel which snapping action is accompanied by an audible signal and a sensitive sensing signal acting against the patient's heel.

The Hida et al. patent (U.S. Pat. No. 7,189,931 B2) discloses a seat occupant load sensor for measuring a seat load from the seat side in a vertical direction to attain the greatest possible accuracy of detection.

The Haselhurst et al. patent (U.S. Pat. No. 7,191,644 B2) discloses a gait assistive system that includes a removable insole that is placed inside the shoe for proportionally sensing touchdown of the limb.

The Vogel et al. patent (U.S. Pat. No. 7,214,202 B2) discloses therapeutic apparatus for treating ulcers by employing intermittent pneumatic compression therapy and vacuum assisted closure therapy that is applied to a wound on the patient's foot.

The Ellis, III, patent (U.S. Pat. No. 7,334,350 B2) discloses a removable midsole structure that includes a removable midsole section that is non-orthotic and can include a compartment having a fluid, a flow regulator, and a control system including a microprocessor.

Nonetheless, despite the ingenuity of the above-described systems and devices, there remains a need for device that allows both the patient and the care giver (therapist, nurse, physician, etc.) to accurately visualize the correct pound pressure (weight bearing) that is being applied to and exerted on the involved extremity by the patient while standing or during ambulation as part of the gait training recuperation and rehabilitation process.

### SUMMARY OF THE INVENTION

The present invention comprehends a medical device that accurately measures pounds pressure as the patient, post-surgically recuperating from a lower extremity injury and/or surgery, stands or ambulates during the rehabilitation process. The medical device is used in physical rehabilitation for individuals learning to walk again after a lower extremity injury and/or surgery, such as a hip replacement, or by those suffering from diabetes and other ailments and conditions that their ability to ambulate.

The present invention comprehends a flexible padded elongated foot wrap with the wrap defined by opposed ends that overlap and attach to each other for securing the wrap on and about the patient's foot. The wrap further defines a continuous outer surface and an opposite continuous inner surface with the continuous inner surface adjacent to and abutting the patient's foot when the wrap is disposed thereon. The ends of the wrap include hook and loop fastening material for securing the ends together, and the hook and loop fastening material can also cover a substantial portion of the continuous outer surface and the continuous inner surface for providing even more surface area for securing the wrap on and about the patient's foot.

The medical device includes a number of features and elements that provides the caregiver and the patient with immediate (real time) visual feedback on the exact amount of pressure—in pounds or kilograms—that the patient exerting so that the correct and prescribed amount of pressure is exerted thereby avoiding the problem of the patient exerting too much pressure on the lower extremity and thus interfering with, impeding and setting back his or her recuperation and recovery.

Thus, the medical device includes a flat beam load cell sensor contained within the wrap and protected by a plastic cap that allows access to the sensor should it ever be damaged. The electrical wires extend out from the sensor to a digital display located on the upper surface of the wrap. The digital display includes a flat flexible plastic film screen on which a digital readout is produced with the digital readout in oversize black indicia (letters and numbering) on a white background. The digital readout is preferably LCD generated, and power for the digital display comes from a flat lithium nickel size battery preferably a CR 2032 that is electrically interconnected to the digital display and is housed in a battery compartment having a removable battery cover. When the wrap is placed on and about the patient's foot, the load cell sensor is positioned in or at the center of the ball of the patient's foot and the digital display is located on the upper part of the patient's foot whereupon the black indicia are easily viewable by both the patient and the caregiver.

It is an objective of the present invention to provide a medical pressure measuring device that accurately measures pounds pressure as a patient attempts to stand or ambulate after having a surgery for a lower extremity such as for hip fractures or hip replacement.

It is another objective of the present invention to provide a medical pressure measuring device that allows both the caregiver and the patient to easily and accurately discern the correct pound pressure that is being exerted by the patient while standing and ambulating and during gait training in general.

It is still another objective of the present invention to provide a medical pressure measuring device that is specifically targeted for post-operative and post-injury rehabilitation and recuperation of the lower extremities.

It is yet another objective of the present invention to provide a medical pressure measuring device that is cost effective, lightweight, portable, fits over the patient's sock and is available in a range of sizes.

It is still another objective of the present invention to provide a medical pressure measuring device that allows patients with diabetic neuropathy, peripheral vascular disease or altered sensory perception with an easily visually discernible and accurate weight bearing status readout in real time.

It is still yet another objective of the present invention to provide a medical pressure measuring device that can be used by the patient as part of his or her out patient physical therapy routine or program.

Still another objective of the present invention is to provide a medical pressure measuring device that utilizes hook and loop fastening arrangement that allows for the quick and easy attachment to and detachment from the foot of the patient.

Still yet another objective of the present invention is to provide a medical pressure measuring device that removes the guesswork from determining the amount of weight bearing pressure, in pounds or kilograms, that is being exerted by the patient during standing and ambulation.

Still yet a further objective of the present invention is to provide a medical pressure measuring device that can be sized for both children and adults and is easily adjustable for fitting onto or over various types of foot coverings such as socks, slippers, shoes, etc.

A further objective of the present invention is to provide a medical pressure measuring device that instantly registers the exact and accurate pound (kilogram) pressure upon the exertion of weight by the patient thereby obviating the need to tap one's foot to initiate the visual activation of the device.

These and other objects, features, and advantages will become apparent to those skilled in the art upon a perusal of the following detailed description read in conjunction with the accompanying drawing figures and appended claims.

### BRIEF DESCRIPTION OF THE DRAWINGS

FIG. 1 is a top plan view of the medical pressure measuring device of the present invention illustrating the digital display in both pounds and kilograms;

FIG. 2 is a bottom plan view of the medical pressure measuring device of the present invention illustrating the plastic cap that covers and protects the flat beam load cell sensor for the device;

FIG. 3 is a perspective view of the medical pressure measuring device of the present invention illustrating the device wrapped about both feet of a patient upright, ambulating and supporting himself/herself on support bars and applying the specified amount of weight bearing on the lower extremities down through the feet for registration and measurement by the device;

FIG. 4 is a perspective view of the medical pressure measuring device of the present invention illustrating the disposition of the device on the foot of the patient with the flat beam load cell sensor located at the center of the ball of the foot of the patient;

FIG. 5 is an enlarged partial top plan view of the medical pressure measuring device of the present invention illustrating a read-out on the flexible plastic screen of the digital display in kilograms;

FIG. 6 is an enlarged partial top plan view of the medical pressure measuring device of the present invention illustrating a read-out on the flexible plastic screen of the digital display in English pounds;

FIG. 7 is a schematic of the medical pressure measuring device of the present invention illustrating the basic operational steps of the device; and

FIG. 8 is a front elevational view of a laminated weight scale chart for the medical pressure measuring device that provides visual guidance to the patient and physician/physical therapist/caregiver to obtain the correct pressure to be exerted by the patient through his/her extremities during static standing and ambulation.

### DETAILED DESCRIPTION OF THE PREFERRED EMBODIMENT

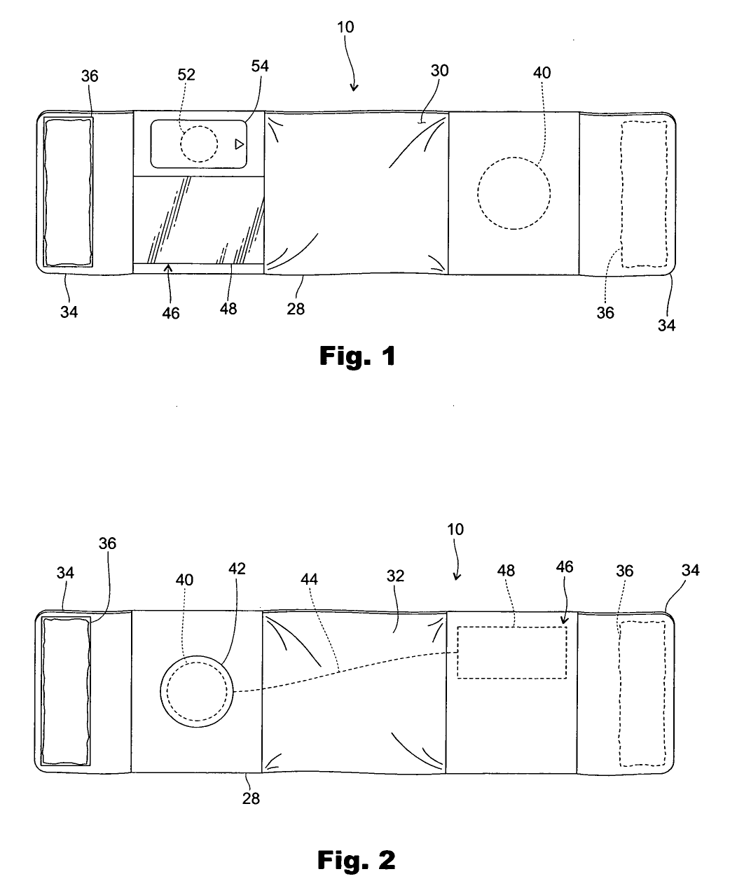

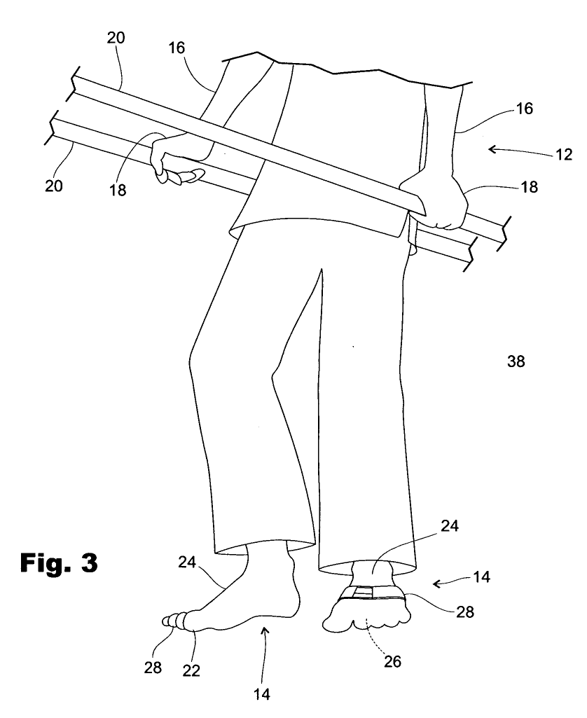

Illustrated in FIGS. 1-8 is a lightweight, portable medical pressure measuring device 10 for accurately measuring pressure, either in pounds or kilograms, exerted by a patient while standing or ambulating during a rehabilitative physical therapy session that is under the careful attention and supervision of a doctor and/or physical therapist. Such physical therapy sessions are for individuals' that need to learn to walk again after surgery for lower extremity fractures or injuries such as hip replacements or hip fractures. Thus, shown in FIG. 3 is a standing individual 12 with one medical pressure measuring device 10 wrapped about each foot 14 of the individual 12. The individual 12 has his/her arms 16 down by his/her side so that the individual's 12 hands 18 can grasp parallel spaced-apart assist bars 20 that the individual 12 traverses by walking in between the assist bars 20 from one end of the assist bars 20 to the other end of the assist bars 20. The measuring device 10 is wrapped about the sole 22 and the upper side 24 of the foot 14 with the readable features of the device 10 situated on the upper side 24 of the foot 14 and the pressure sensing element positioned at the underside of the foot 14 and more specifically at the center of the ball 26 of the foot 14.

Thus, as shown in FIGS. 1-6, the medical pressure measuring device 10 includes a flexible, elongated foot wrap 28 with one preferable dimension of the wrap 28 being 16 inches long by three to three and ½ inches wide. The wrap 28 comprises a padded hook and loop fastener arrangement and is configured similar to blood pressure cuff material. The wrap 28 further defines a continuous outer surface 30 and an opposite continuous inner surface 32 with the continuous inner surface 32 adjacent and abutting the patient's foot 14. The opposed ends 34 of the wrap 28 include a securement strip or portion 36 of hook and loop fastening material; and as shown more particularly in FIGS. 1 and 2, one securement strip 36 is located on the outer surface 30 adjacent one end 34 of the wrap 28 and the other securement strip 36 is located on the inner surface 32 adjacent the other end 34 of the wrap 28 opposite the securement strip 36 on the outer surface 30. The ends 34 of the wrap 28 are brought together so that the securement strips 36 can be attached to each other for securing the opposed ends 34 of the wrap 28 to each other and thus securing the wrap 28 on and about the foot 14 of the individual 12. When the wrap 28 is placed on the foot 14 of the individual 12 the wrap 28 further defines an upper side 24 and an opposite under side that comprises and includes the ball 26 of the foot 14. In an alternative embodiment the hook and loop fastening strip can be configured and extended to cover a substantial portion of both the outer surface 30 and the inner surface 32 for achieving an even firmer securement of the wrap 28 to itself and also on and about the foot 14 of the individual 12.

As shown in FIGS. 1-7, the medical device 10 of the present invention includes a number of elements and features that provide the patient and the health care specialist or supervisor with immediate (real time) easily discernible visual feedback of the amount of pressure, in pounds or kilograms, being exerted by the patient 12 when static standing or during ambulation usually with an assist device such as the spaced parallel bars or walkers and crutches. The medical device 10 of the present invention eliminates the guesswork in determining that the correct amount of pressure is being exerted by the patient 12 during standing or ambulation. This eliminates the problem of the patient 12 exerting too much pressure on and through the lower extremities 38 (legs, thighs, calves, ankles, etc.) and thus interfering with, and possibly impeding and setting back, the recuperation and rehabilitation process.

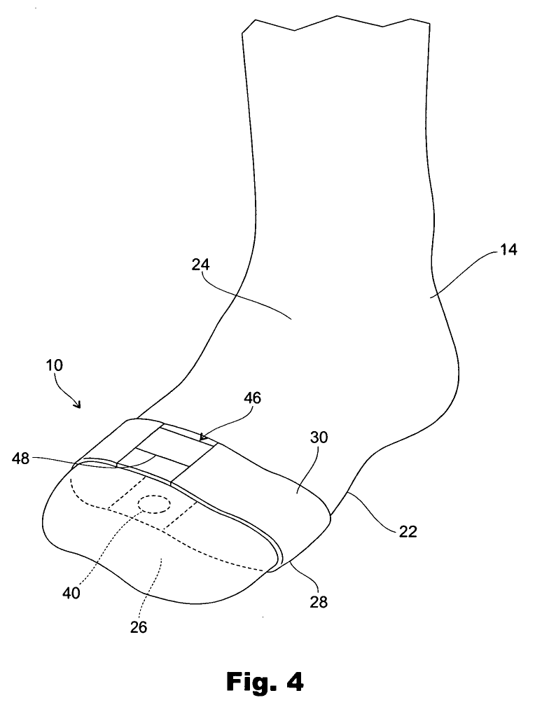

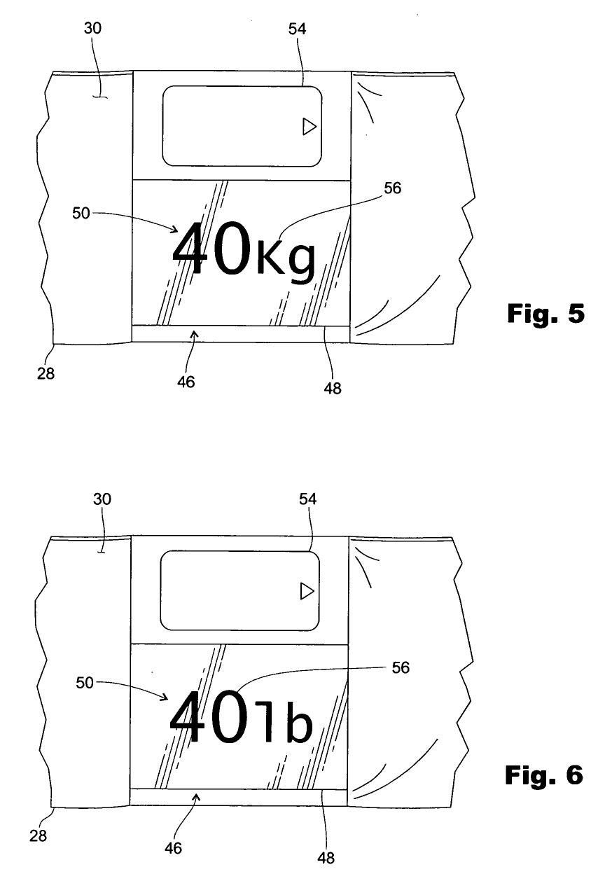

Thus, as shown in FIGS. 1, 2, 4 and 7, the pressure sensing element for the medical device 10 includes a flat beam load cell sensor 40 contained within the wrap 28 and that is protected by a plastic cap 42 that allows access to the load cell sensor 40 should it need replaced. The load cell sensor 40 is positioned on the underside of the wrap 28 when the wrap 28 is placed on the individual's foot 14, and, more specifically, the load cell sensor 40 is positioned adjacent to and immediately beneath the center ball 26 of the individual's foot 14. Electrical wires 44 extend out from the load cell sensor 40 to a digital display 46 located on the upper side of the wrap 28 and which is easily visible and discernible for the patient 12 and the physical therapist assisting and supervising the patient 12. The digital display 46 includes a flat flexible plastic film screen 48 on which a digital readout 50 appears to register the amount of pressure being exerted by the individual 12. The digital readout 50 is preferably LCD generated, and power for the digital display 46 and the digital readout 50 comes from a coin size battery 52 electrically interconnected to the digital display 46 and which is housed in a battery compartment having a removable battery cover 54 for access thereto. The battery 52 preferably is a flat lithium nickel size battery preferably a CR 2032 that is widely used in watches, keyless entries, calculators, cameras, toys, etc. The digital readout 50 is in the form of oversize black indicia (letters) 56 on white background. FIG. 4 illustrates the digital readout 50 wherein the indicia 56 display the readout of “40 kg” and FIG. 5 illustrates the digital readout 50 wherein the indicia 56 display the readout of “40 lb.” In addition, the digital readout 50 should be able to visually register at least 200 pounds/kilograms of pressure exerted and the indicia 56 displayed on the plastic film screen 48 are preferably three inches by one and ½ inches.

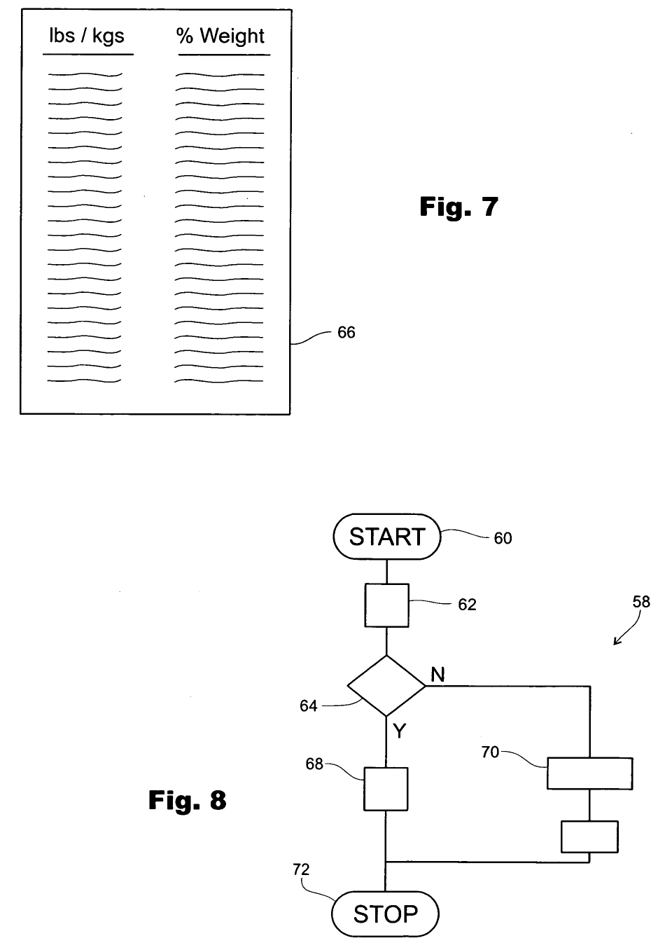

FIG. 7 illustrates a schematic flowchart 58 of the steps the patient 12 and caregiver undertake to achieve the correct pressure as a percentage of the patient's body weight to be exerted by the patient 12 during static standing and ambulation. In FIG. 7 the process starts—start process 60—when the wrap 28—wrap placement 62—is placed on the foot 14 of the individual 12. The individual 12 then exerts downward force (pressure) on the foot 14 through the extremities 38 with the digital display 46 immediately showing the readout 50 in pounds or kilograms of the pressure being exerted. A therapeutic decision 64 occurs next: if the amount of pressure being exerted is correct with respect to the patient's 12 body weight as prescribed by the physician and set forth on a laminated scale chart 66 the patient 12 continues exerting that amount of pressure—maintain pressure 68. However, if the patient 12 is not exerting the appropriate and correct amount of pressure, the digital readout 50 will immediately display that pressure and then pressure modification and adjustment 70 can be made to bring the pressure being exerted within the correct range or amount. The wrap 28 can then be removed when the therapy session reaches an end or stop 72. Also, FIG. 8 illustrates the portable laminated weight chart or scale chart 66 that comes with the medical device 10. The scale chart 66 is designed to eliminate the guesswork by the patient 12 and caregiver in determining the exact amount of pressure that can be and is to be exerted on and through the extremities 38 to the feet 14 during the patient's 12 rehabilitation process.

While the invention has been described with respect to a preferred embodiment, it will be manifest to those skilled in the art that numerous alterations, modifications, and variations are possible and practicable while still remaining within the ambit and scope of the invention as set forth in the detailed description of the preferred embodiment of the invention and the appended claims.

1. A medical pressure measuring device for registering the amount of pressure exerted by a patient through the extremities and to the feet when standing and ambulating, comprising:
a flexible elongated foot wrap including:
a continuous outer surface and an opposite continuous inner surface;
a pair of opposed ends;
a pair of securement strips with one securement strip located on the outer surface adjacent one end and the other securement strip located on the inner surface adjacent the other end whereby the securement strips are brought together for securing the wrap on and about the foot of the patient;
a digital display located on the continuous outer surface for displaying a digital readout of the amount of pressure being exerted by the patient;
a load cell sensor disposed within the wrap electrically interconnected to the digital display for sensing the amount of pressure being exerted by the patient;
a battery disposed within the wrap and electrically interconnected to the digital display for powering the digital display; and
the load cell sensor for continuously sensing the amount of pressure being exerted by the patient during standing and ambulation and the digital display continuously displaying the amount of pressure being exerted so that the patient can adjust the amount of pressure being exerted to achieve the correct pressure exerted through the extremities.

2. The medical pressure measuring device of claim 1 wherein the digital display includes a flat flexible plastic screen.

3. The medical pressure measuring device of claim 2 wherein the digital readout shown on the digital display is in pounds.

4. The medical pressure measuring device of claim 3 wherein the digital readout shown on the digital display is in kilograms.

5. The medical pressure measuring device of claim 4 wherein the digital readout includes indicia in the form of black letters.

6. A medical pressure measuring device for registering the amount of pressure exerted by a patient through the extremities and to the feet when standing and ambulating, comprising:
a flexible elongated foot wrap including:
a continuous outer surface and an opposite continuous inner surface;
a pair of opposed ends;
a pair of securement strips with one securement strip located on the outer surface adjacent one end and the other securement strip located on the inner surface adjacent the other end whereby the securement strips are brought together for securing the wrap on and about the foot of the patient;
a digital display located on the continuous outer surface for displaying a digital readout of the amount of pressure being exerted by the patient;
the digital display including a flat flexible plastic screen;
a load cell sensor disposed within the wrap electrically interconnected to the digital display for sensing the amount of pressure being exerted by the patient;
a battery disposed within the wrap and electrically interconnected to the digital display for providing power for the digital display; and
the load cell sensor for continuously sensing the amount of pressure being exerted by the patient during standing and ambulation and the digital display continuously displaying the amount of pressure being exerted so that the patient can adjust the amount of pressure being exerted to achieve the correct pressure that should be exerted through the extremities.

7. The medical pressure measuring device of claim 6 wherein the digital readout shown on the digital display is in pounds.

8. The medical pressure measuring device of claim 7 wherein the digital readout shown on the digital display is in kilograms.

9. The medical pressure device of claim 8 wherein the digital readout includes indicia in the configuration of black letters.
